(19) United States
(12) Patent Application Publication
(10) Pub. No.: US 2016/0207022 A1
(43) Pub. Date: Jul. 21, 2016

(54) DNA MILLICHIP

(71) Applicant: WISCONSIN ALUMNI RESEARCH FOUNDATION, Madison, WI (US)

(72) Inventors: Michael R. Sussman, Cross Plains, WI (US); Francesco Cerrina, Boston, MA (US); Kathryn Richmond, Madison, WI (US); Jamison Wolfer, Madison, WI (US)

(21) Appl. No.: 15/090,306

(22) Filed: Apr. 4, 2016

Related U.S. Application Data
(63) Continuation of application No. 12/474,747, filed on May 29, 2009.
(60) Provisional application No. 61/057,478, filed on May 30, 2008.

(51) Int. Cl. B01J 19/00; C12Q 1/68

(52) U.S. Cl. CPC B01J 19/0046; C12Q 1/6837; B01J 2219/00596; B01J 2219/00722; B01J 2219/00436; B01J 2219/00608; B01J 2219/00529

(57) ABSTRACT

The present invention provides novel arrays of oligonucleotide probes immobilized on a solid support in the form of a chip (millichip), which can be used for rapid and inexpensive analysis of nucleic acids. The arrays can have a plurality of different oligonucleotide probes that can provide for whole genome gene expression analysis. The millichip can be used for analysis of both RNA and DNA.

### CROSS-REFERENCE TO RELATED APPLICATIONS

This application is a continuation of U.S. patent application Ser. No. 12/474,747, filed May 29, 2009, which claims priority to U.S. Provisional Patent Application No. 61/057,478, filed May 30, 2008, the entirety of both of which are incorporated herein by reference.

### GOVERNMENT INTERESTS

This invention was made with United States government support under HG003275 awarded by the National Institutes of Health and 0448369 awarded by the National Science Foundation. The government has certain rights in the invention.

### FIELD OF THE INVENTION

The present invention provides arrays of immobilized oligonucleotide probes for analyzing molecular interactions of biological interest, and therefore relates to diverse fields impacted by the nature of molecular interaction, including chemistry, biology, medicine, and medical diagnostics.

### BACKGROUND

An unpleasant fact of life for current genomic studies is the very high cost of obtaining whole genome RNA measurements. Despite the great potential of DNA (gene) chips containing arrays of oligonucleotides for revolutionizing the study of biology (Ramsay, 1998, I 16: 40-44; U.S. Pat. No. 5,837,832), the greatest limitation to the widespread use of this technology is its cost. At several hundreds of U.S. dollars for each DNA chip, as well as the several hundreds of U.S. dollars cost for the labeling reactions, and the need to perform at least several (e.g. quadruplicate) biological replicates per treatment, the cost alone prevents widespread use by laboratories, on a daily basis. The time and equipment needed for analyses are significant limiting factors as well.

Recently, an instrument was created that synthesizes high density Affymetryx-style (Affymetryx, Santa Clara, Calif.) oligonucleotide chips, but without the expense and time needed to synthesize chromium masks for each sequence set (each mask can cost approximately $1,000 and take weeks to manufacture, and approximately 100 different masks are needed for each set of 25-mers). This instrument, called a maskless array synthesizer (MAS), can be readily built from conventional engineering industry parts, as described by Singh-Gasson et al. 1999, Nature Biotechnology 17: 974-978. The MAS is basically a benchtop machine that creates a microscope slide containing hundreds of thousands and in more recent derivations, millions of different oligonucleotides, covalently attached every 4-6 hours, depending on length. While the Affymetrix mask method is generally limited to short, 20-25 bp long oligonucleotides, the MAS readily makes much longer oligos, an important aspect of the millichip technology described herein. The technology underlying this invention was commercialized by NimbleGen Systems, Madison, Wis.

Although the one time cost of approximately $100,000 for building a single maskless array synthesizer, with current oligo density of about 1.2 million oligos per 2 cm² glass surface, is not a large investment for such instruments, the cost per chip remains a significant problem due to the significant cost of DNA synthesis reagents. For example, the cost of phosphoramidites with light-sensitive protecting groups at their 3′ hydroxyl end required per chip is approximately $100. Since one can grid ten thousand oligonucleotide solutions (with a feature size of about 50-100 μm diameter per oligo) onto microscope slides for less than $50 per chip in consumable reagents, once the investment of approximately $50,000 needed to buy a set of 10,000 different 70-mer oligonucleotides is made, it is likely that if one does not have access to a MAS instrument, gridded arrays using a robotic gridder is a cheaper and more cost effective means of performing large amounts of hybridizations. However, when one considers the labor involved in maintaining and operating the robotic gridder, the cost of such chips, along with the RNA labeling reagents needed for the hybridizations on the several cm squared surfaces, brings the total cost per single hybridization to over $100. This precludes being able to use gridded chips to perform studies of multiple treatments or conditions, in sufficient number of replicas.

Overcoming a major genomic roadblock can be achieved through the development of an improved array of oligonucleotide probes, such as an, inexpensive DNA chip that can be widely used for low-cost, high throughput whole genome gene expression analysis. Preferably, the DNA chip can be assayed by investigators using significantly less effort and cost in comparison to current methods that typically require capital equipment. The present invention provides these and related needs.

### BRIEF SUMMARY

Arrays of oligonucleotide probes immobilized on solid supports are provided. The arrays include at least about 1,000 different oligonucleotide probes and no more than about 100,000 different oligonucleotide probes that are about 30 to about 100 nucleotides in length. The oligonucleotide probes occupy separate known sites in the arrays. The arrays have density of at least about 500,000 oligonucleotide probes per 1 cm² solid support surface. The volume of the solid support is between about 0.125 mm³ and about 30 mm³.

In one embodiment, the arrays of the present invention include at least two sets of oligonucleotide probes: (1) a first set of oligonucleotide probes that is exactly complementary to a set of reference sequences; and (2) a second set of oligonucleotide probes that is identical to the first set of oligonucleotide probes but for at least one different nucleotide. In another embodiment, the arrays of the present invention include at least two sets of oligonucleotide probes: (1) a first set of oligonucleotide probes that is exactly complementary to a set of reference sequences; and (2) a second set of oligonucleotide probes that is a reverse-complement to the first set of oligonucleotide probes. The arrays may include a set of oligonucleotide probes that is complementary to a genome.

In one example of the arrays of the present invention, the oligonucleotide probes are about 70 nucleotides in length. In one example of the arrays of the present invention, the density of the arrays is about 610,000 oligonucleotide probes per 1 cm² solid support surface. Also, in one example of the arrays of the present invention, the volume of the solid support is about 8 mm3.

The oligonucleotide probes may be covalently attached to the solid support. The oligonucleotide probes may be oligodeoxyribonucleotides. The solid support may be an article that includes at least one of a porous substrate, a non-porous substrate, a three-dimensional surface, and a planar surface.

Methods for making arrays of oligonucleotide probes are provided. The methods include immobilizing on solid support arrays that comprise at least 1,000 different oligonucleotide probes and no more than about 100,000 different oligonucleotide probes that are about 30 to about 100 nucleotides in length, the oligonucleotide probes occupying separate known sites in the arrays, the arrays having density of at least 500,000 oligonucleotide probes per 1 cm² solid support surface. The volume of the solid support is between about 0.125 mm³ and about 30 mm³.

In one embodiment, the methods include immobilizing on the solid support arrays that comprise at least two sets of oligonucleotide probes: (1) a first set of oligonucleotide probes that is exactly complementary to a set of reference sequences; and (2) a second set of oligonucleotide probes that is identical to the first set of oligonucleotide probes but for at least one different nucleotide. In another embodiment, the methods include immobilizing on the solid support arrays that comprise at least two sets of oligonucleotide probes: (1) a first set of oligonucleotide probes that is exactly complementary to a set of reference sequences; and (2) a second set of oligonucleotide probes that is a reverse-complement to the first set of oligonucleotide probes. The methods may be practiced where the arrays include a set of oligonucleotide probes that is complementary to a genome.

In one example of the methods of the present invention, the oligonucleotide probes are about 70 nucleotides in length. In one example of the methods of the present invention, the density of the array is about 610,000 oligonucleotide probes per 1 cm² solid support surface. In one example of the methods of the present invention, the volume of the solid support is about 8 mm³.

The oligonucleotide probes may be covalently attached to the solid support. The oligonucleotide probes may be oligodeoxyribonucleotides. The solid support may be an article that includes at least one of a porous substrate, a non-porous substrate, a three-dimensional surface, and a planar surface.

Methods for detecting expression of a plurality of genes are provided. The methods include: a) providing an array of oligonucleotide probes immobilized on a solid support, where the array includes at least 1,000 different oligonucleotide probes and no more than about 100,000 different oligonucleotide probes that are about 30 to about 100 nucleotides in length, the oligonucleotide probes occupying separate known sites in the array, the array having density of at least 500,000 oligonucleotide probes per 1 cm² solid support surface, the solid support having volume of between about 0.125 mm³ and about 30 mm³; b) hybridizing a labeled sample nucleic acid target to the array of oligonucleotide probes; and c) measuring the label intensity to identify the level of gene expression for each of the labeled sample nucleic acid targets bound to complementary oligonucleotide probes. The methods of the present invention may be practiced where the plurality of genes comprise a genome. In one example, the methods may be practiced where the hybridization is performed in about 5 μl to about 15 μl of hybridization solution. The methods may be practiced where detecting the expression of a plurality of genes is used to identify genetic polymorphisms.

### BRIEF DESCRIPTION OF THE DRAWINGS

FIGS. 1(A, B) depicts images of a 1 mm thick microscope slide used as solid substrate for array, and subdivided into 20 squares (i.e., 4×5).

FIGS. 2(A, B) depicts images of a subdivided (1 mm×3 mm×3 mm) millichip and its hybridization.

FIG. 3 depicts images of hybridized arrays utilizing varying cut depths (0-0.7 mm cut depth) prior to subdivision.

FIG. 4 depicts four composite fluorescent microscope images that are required for each millichip, in accord with one embodiment of the present invention.

FIG. 5 is a fluorescence scanner image of control slide millichip area.

### DETAILED DESCRIPTION OF THE PRESENTLY PREFERRED EMBODIMENTS

This invention relates generally to the field of biology, and particularly to techniques for the analysis of nucleic acids using arrays. In one aspect, the present invention provides compositions and methods for making DNA chips relatively easy and cheap, so that the DNA chip technology can become a common part of many laboratories. In one preferred embodiment, the present invention provides a novel array of oligonucleotide probes in the form of a DNA chip, called “millichip”. With millichips of the present invention, the cost for performing whole genome expression studies can be reduced over a hundred fold, and the time needed for completion of experiments can be reduced substantially, perhaps as much as ten-fold.

Generally, the nomenclature and the laboratory procedures described below are those well known and commonly employed in the art. Standard techniques are used for cloning, DNA and RNA isolation, amplification and purification. Enzymatic reactions involving DNA ligase, DNA polymerase, restriction endonucleases and the like are generally performed according to the manufacturer's specifications. These techniques and various other techniques are generally performed according to Sambrook et al. 1989, Molecular Cloning—A Laboratory Manual, Cold Spring Harbor Laboratory, Cold Spring Harbor, N.Y.; Ausubel et al., 1993, Current Protocols in Molecular Biology, Volumes 1-3, John Wiley & Sons, Inc., New York, N.Y.; and Kriegler, 1990, Gene Transfer and Expression: A Laboratory Manual, Stockton Press, New York, N.Y., each of which is incorporated herein by reference in its entirety.

The term “MAS” refers to a “maskless array synthesizer”, such as the one using the technology described by NimbleGen Systems, Madison, Wis. (Singh-Gasson et al., 1999, Nature Biotechnology 17: 974-978). A “MAS derived microscope slide” refers to a microscopic slide on which an array of oligonucleotide probes has been synthesized using a maskless array synthesizer.

A “genome” of an organism is its whole hereditary information and is encoded in the DNA (or, for some viruses, RNA). A “genome” refers to a complete DNA sequence of one set of chromosomes, and is meant to include the complete set of nuclear DNA (i.e., the “nuclear genome”) but can also be applied to organelles that contain their own DNA, as with the mitochondrial genome or the chloroplast genome.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or proteins for which antisera or monoclonal antibodies are available. The present invention contemplates the use of labeled nucleic acids, such as labeled RNA or labeled DNA.

The terms “oligonucleotide probes”, “oligomers”, or “oligos” are used for short, single stranded DNA fragments, generally used as probes in hybridization experiments (such as oligos bound to glass surfaces or nylon membranes). The length of the oligos in the practice of the present invention can vary from about 30 DNA monomer units (i.e., 30-mers or 30-mer oligonucleotide probes) to about 100 DNA monomer units (i.e., 100-mers or 100-mer oligonucleotide probes). In one embodiment, the oligomers used in the practice of the present invention are 50-mers (i.e., 50-mer oligonucleotide probes). In yet one embodiment, the oligomers used in the practice of the present invention are 70-mers (i.e., 70-mer oligonucleotide probes). Longer oligonucleotide probes enable stronger hybridization, i.e. hybridization using higher stringency conditions, as described below. Hybridization using higher stringency conditions provides higher specificity and reduces non-specific binding.

The oligonucleotide probes are typically immobilized on some type of solid support. The type of solid support can vary. For example, the solid support can preferably be an article that includes one or more of porous substrates, non-porous substrates, three-dimensional surfaces, beads, planar surfaces, etc. Generally, what is important for practicing the present invention is that the solid support provides for a surface on which relatively high density of oligonucleotide probes can be attached. The oligonucleotide sequences can be immobilized to the solid support using covalent attachments. Alternatively, or in addition, the oligonucleotide sequences can be immobilized to the solid support via some type of a linker known in the art.

Preferably, the oligonucleotide probes are immobilized on the solid support in the form of an array with a relatively high density. In one embodiment, array with preferred density of oligonucleotide probes immobilized on the solid support include arrays that have a relatively large density of preferably at least 500,000 oligonucleotide probes per 1 cm² solid support surface. The relatively high density can be achieved using ultra high density oligo array technology. For example, this can be conveniently achieved with a maskless array synthesizer with spots as small or smaller than 10 micrometers, whereas the gridded arrays require a pen that can deposit a spot with approximately 100 micrometers in diameter and thus cannot achieve this density made with the MAS. The oligonucleotide probes thus occupy separate known sites in the array.

Arrays of oligonucleotide probes immobilized on solid supports are provided. The arrays include at least 1,000 different oligonucleotide probes, preferably at least 10,000 different oligonucleotide probes, more preferably at least 50,000 different oligonucleotide probes, and preferably no more than about 100,000 different oligonucleotide probes that are about 10 to about 100 nucleotides in length, preferably about 20 to about 80 nucleotides in length, and more preferably about 30 to about 70 nucleotides in length. The oligonucleotide probes occupy separate known sites in the arrays. The arrays have density of at least 300,000 oligonucleotide probes per 1 cm² solid support surface, preferably the arrays have density of at least 500,000 oligonucleotide probes per 1 cm² solid support surface, and more preferably the arrays have density of at least 1,000,000 oligonucleotide probes per 1 cm² solid support surface. The volume of the solid support is between about 0.05 mm³ and about 30 mm³, preferably the volume of the solid support is between about 0.1 mm³ and about 20 mm³, and more preferably the volume of the solid support is between about 0.2 mm³ and about 10 mm³.

In some embodiments of the present invention, the oligonucleotide probes are preferably immobilized in the form of sets. For example, a set of oligonucleotide probes can be exactly complementary to a set of reference sequences, e.g. to a known genome. Another set of oligonucleotide probes can be identical to the first set of oligonucleotide probes but for at least one different nucleotide (i.e., one or more oligonucleotide probes can be modified to provide a desired mismatch vs. the set of reference sequences). Another set of oligonucleotide probes can be exactly identical to a set of reference sequences. Yet another set of oligonucleotide probes can be a reverse-complement to a set of known reference sequences. A variety of combinations of the above sets of arrays can also be immobilized on solid support, to provide a medley of various oligonucleotide probes that can be used for probing different properties of the complementary labeled polynucleotides that are hybridized to the array, as described below.

Oligonucleotide probes may be synthesized using a variety of methods. Suitably, arrays of DNA sequences used as probes may be synthesized using the apparatus and method disclosed in U.S. Pat. No. 7,037,659 B2, which is incorporated herein by reference. As well, arrays of DNA probes on a surface of a substrate can be synthesized using the apparatus and method disclosed in U.S. Pat. No. 6,375,903 B1, which is incorporated herein by reference. Using the arrays and the methods of the present invention, it is possible to accomplish DNA synthesis onto the uncut and pre-scored slide surface. Successful hybridization was seen regardless of whether DNA synthesis was performed pre- or post-cutting of the slide into millichip(s), indicating that flexibility exists with the timing of the cutting step.

The invention also relates to nucleic acids that selectively hybridize to the exemplified oligonucleotide sequences, including hybridizing to the exact complements of these sequences. The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions.

To hybridize under “stringent conditions” describes hybridization protocols in which nucleotide sequences in the two strands are at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

“Stringent hybridization conditions” are conditions that enable a probe, primer or oligonucleotide to hybridize only to its target sequence. Stringent conditions are sequence-dependent and will differ. One stringent condition example comprises hybridization in 1M [Na+], 100 mM IVIES, 20 mM EDTA, and 0.01% Tween-20 at 45° C., with washes at 45° C. in 6×SSPE, 0.01% Tween-20, followed by a high-stringency wash consisting of 100 mM MES salt and free acid solution, 0.1M [Na+], 0.01% Tween-20. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

“Moderately stringent conditions” use washing solutions and hybridization conditions that are less stringent (Sambrook et al., 1989), such that a polynucleotide will hybridize to the entire, fragments, derivatives, or analogs of a given polynucleotide sequence, e.g. SEQ ID NO:1. One example comprises hybridization in 6×SSC, 5×Denhardt's solution, 0.5% SDS and 100 m/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described in the art (Ausubel et al., 1993; Kriegler, 1990).

“Low stringent conditions” use washing solutions and hybridization conditions that are less stringent than those for moderate stringency (Sambrook et al. 1989), such that a polynucleotide will hybridize to the entire, fragments, derivatives, or analogs of a given oligonucleotide sequence. A nonlimiting example of low stringency hybridization conditions includes hybridization in 35% formamide, 5×SSC, 50 mM Tris HCl (pH 7.5), 5 mM EDTA, 0.02% PVP, 0.02% Ficoll, 0.2% BSA, 100 m/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency, such as those for cross species hybridizations are well-described (Ausubel et al., 1993; Kriegler, 1990).

In one example, the components of the millichip include about 5,000 to about 30,000 different 70-mer DNA oligonucleotide sequences (oligos), each oligo covalently attached to an about 2 μm2-15 μm2 glass surface, with the whole set of about 5,000 to about 30,000 70-mers collectively placed on an about 2.25 mm3 (about 1.5 mm×about 1.5 mm on the surface, and about 1 mm thick) piece of glass (used as solid support), which is delivered and utilized for the experiments, in the bottom of a small microfuge tube (herein also called microcentrifuge, eppendorf) tube. This can be, e.g., a 0.5 ml plastic microfuge (microcentrifuge) tube. In one embodiment, the hybridizations are performed in relatively low volumes, e.g. about 5-10 microliter volumes in a microfuge tube, surrounding and bathing the about 2.25 mm3 piece of glass, and the readout of labeled nucleic acid (e.g. labeled RNA) concentrations hybridizing to the chip is performed by routine fluorescence microscopy, using for example a confocal microscope. In one embodiment, the oligonucleotides are immobilized on one surface of a three-dimensional solid support. In another embodiment, the oligonucleotides are synthesized on more than one surface of a three-dimensional solid support. For example, if the solid support is a cube, the probes may be immobilized on one or more of the surfaces of that cube.

In one example, the present invention contemplates the synthesis of about 800,000 70-mers using a MAS, on a microscope slide with a depth (i.e., thickness) of about 1 mm, and then dividing the slide into about 96 (1.5×1.5 mm) glass pieces, each containing about 30,000 70-mer oligonucleotides corresponding to an organism's genome (for example human genome, Arabidopsis genome). When chosen from the proper sequence within a gene, these 70-mers like the gridded chip approach, are long enough to give excellent quantification without having to make a dozen or more separate 25-mer oligos, as typically done in the art, e.g. by Affymetrix (Santa Clara, Calif.). Of course, one will preferably use two or more separate millichips and perform the desired number of biological replicates (e.g., duplicate, triplicate, or quadruplicate experiments), to measure overall reproducibility. The present invention contemplates the whole genome expression analysis for a variety of prokaryotic and eukaryotic organisms.

Subdividing a piece of glass can be performed in a variety of ways, for example, using conventional technology in the silica engineering industry. In one example, in order to divide the glass substrate into smaller sections, the present invention contemplates the use of a simple approach as commonly employed for silicon wafers. The about 1 mm thick glass substrate is scored on the back using an about 0.1 mm thick diamond saw, to a depth equal to between about 0.3 mm to about 0.7 mm. In one embodiment, this requires 6 cuts along the length, and 7 across, to create 42 pieces. The front of the surface, where the synthesis of oligos occurs, remains intact. In order to avoid light scattering at the cuts, it is possible to use an index-matching optical resin to fill the cuts; this resin can be easily removed afterwards with acetone. After synthesis and removal of the resin, synthesis on the glass slide can be scored using a glass scorer. Light pressure is applied along the score lines to break the glass slide in the intended number of squares (42 squares in this example). Vacuum tweezers can then be used to manipulate the samples. Since the number of pixels can be about 1.3 millions, each small piece will have approximately 25,000-30,000 features. Some features will be lost at the position of the cuts but these can b minimized to ensure that there are at least about 24,000 70-mers available on each millichip, to report on the size of a whole genome, such as the whole Arabidopsis genome. If the volume of chip synthesis warrants it, glass substrates pre-scored using laser micromachining can be made. Everything else in the synthesis process will stay the same.

In one example, the present invention contemplates the use of novel, previously unavailable instruments for dense printing of oligonucleotide probes. For example, the newer MAS instruments have a 1.2 million pixel digital micromirror device that replaces the older 786,000 unit MAS. These new instruments enable the “printing” (i.e., immobilization) of about 600,000 oligonucleotide probes per 1 cm² of support surface.

In preferred embodiments, the present invention contemplates that the volume of these novel DNA millichips can be small, typically ranging from about 0.5 cm³ to about 10 cm³. One advantage of such small substrates that carry arrays of immobilized oligonucleotide probes is that it is possible to use small volumes of prehybridization and/or hybridization solutions for analyses. In addition, the arrays that are immobilized on such relatively small substrates can be visualized using instrumentation readily available in research laboratories, such as fluorescence microscopes and/or confocal microscopes.

In one embodiment, these cut, small cube glass pieces can be considered about the size of small Sephadex beads. These glass pieces can be readily suspended in aqueous solution, which facilitates the downstream analytical steps such as prehybridization, hybridization, wash, etc. In one example, one small glass cube (2 mm×2 mm×2 mm, i.e. with volume of approximately 8 mm³) is placed in the bottom of each of 40 microfuge tubes, and then very small volumes (e.g., 10 microliters) of labeled RNA and wash solutions are used, to perform the hybridizations at a ten-fold lower scale than currently needed for that performed with microscope slides or with commercially-available Affymetrix chips, or any other chip (nucleotide array) platform currently known.

With current prices, the final cost per approximately 8 mm³ (about 2 mm² surface area with about 2 mm depth, i.e. thickness) millichip comprising about 30,000 70-mer oligos would be approximately $10. Other experiments not requiring detecting the expression of 30,000 genes could be performed at an even lower cost. For example, if one is interested in measuring the expression of only 3,000 genes for a particular question, one could make 400×2 mm³ (1 mm² area×2 mm depth, i.e. thickness) millichips, in which case the volume used, and thus the cost per hybridization, would be ten fold lower, or approximately $1.

The solid supports of the present invention can take a variety of shapes. For example, the solid supports can be in the form of articles that include at least one of a porous substrate, a non-porous substrate, a three-dimensional surface, a bead, and a planar surface. The solid supports do not have to be equilateral, i.e. they can have asymmetrical sides. For example, solid support in the form of a cube can have three identical sides, e.g. 0.5 mm each (0.5 mm×0.5 mm×0.5 mm), for a volume of 0.125 mm³. Alternatively, solid support in the form of a cylinder can have two identical bases, and a wall of constant circular cross-section.

One advantage of the present invention is that hybridization can be performed in relatively small volumes of hybridization solutions. In one example, careful scaling down of the hybridization volumes is performed, so that reagent savings can be produced commensurate with the reduction in size of the glass surface. The present state of the art typically involves the use of 300 microliters of hybridization solution, which is needed to cover the 2 cm² (400 mm²) surface containing all 786,000 oligos. The millichips of the present invention can have much smaller surface area, e.g. 1/40 the surface area of the slides that are typically used today. Thus, in principle, it is possible to use less than 10 microliters of hybridization solution per whole genome chip. This simplicity and reduction in the amount and cost of labeled RNA reagents and the chips themselves could reduce the cost of each chip experiment from the current approximately $100-$400 (depending on platform and vendor) to approximately $10 or less.

After hybridization of the labeled target and/or reference sequences to the immobilized oligos, signal detection is performed. Detection of the signal can be performed in a variety of ways known in the art. In one example, the millichips are scanned using a standard laboratory confocal microscope, with its routine 2 μm or better resolution, and analyzed with software that allows automatic quantification of the approximately 30,000 fluorescent spots on the 2.5 mm² glass surface. The building and operation of a MAS instrument has already been described by Singh-Gasson et al., 1999, Nature Biotechnology 17: 974-978, which is incorporated herein by reference. Gene expression analysis using oligonucleotide arrays produced by maskless photolithography has also been described by Nuwaysir et al., 2002, Genome Research 12: 1749-1755, which is incorporated herein by reference.

In one embodiment, the present invention utilizes the advantages of controlling at least two of the following variables: (1) length of the oligonucleotide probes; and (2) density of the oligonucleotide probes in the array; (3) volume of solid substrate used for immobilization of the arrays of oligonucleotide probes; (4) volume of prehybridizing and hybridizing solution used. The methods of the present invention provide for the generation of arrays with probes that have optimized oligo length, as well as optimized oligo density. Thus the devices and methods of the present invention provide an unprecedented tool that allows the rapid iterative design and optimization of probes for a variety of applications, such as gene expression profiling and genotyping.

Some Specifics of the DNA Millichip Technology

In one preferred embodiment of the invention, the millichip technology includes four components, which are described as follows.

A small piece of solid substrate, e.g. a 1 mm×1 mm×1 mm cubed piece of glass (volume of 1 mm³), with at least about 5,000 (for yeast) or with at least about 30,000 (for human and Arabidopsis) different oligos, for example 70-mers. These 70-mers can be, for example, derived from the 3′ end of each gene, and can be covalently attached to one or more of the six surfaces of this cube. In addition, or in the alternative, the 70-mers can be attached to the one or more of the six surfaces of this cube via linker using methods known in the art. Instead of the approximately $200 required to synthesize a large microscope slide containing these oligos (oligomers), the present invention provides for the synthesis of 50-200 exact copies of the same 5,000-30,000 70-mers on the 2 cm² glass microscope slide surface using the maskless array synthesizer. The glass microscope slide is then cut into a number of pieces, preferably identical or approximately identical pieces. The number of pieces can vary, and in one embodiment it is about 50 to about 200 identical pieces, each one representing a whole genome expression array (e.g., about 5,000 70-mers are used for yeast; about 30,000 70-mers are used for human or Arabidopsis). Oligos can be synthesized before or after subdivision of the slide into pieces.

In one example, the MAS-derived microscope slide containing about 1.2 million 70-mers can be cut into 49 pieces, each with dimensions of approximately 1 mm×3 mm×3 mm. This can be done, for example, with seven vertical and seven horizontal cuts. Alternatively, the slide can be cut into 225 pieces, each with dimensions of approximately 1 mm×1 mm×1 mm, with 15 vertical cuts and 15 horizontal cuts. Any number of horizontal and vertical cuts can be utilized. The number of horizontal cuts does not have to be the same as the number of vertical cuts. Obviously, by varying the number of vertical and horizontal cuts on the typically used 2 cm squared surface (2 cm²) of the MAS derived microscope slide, millichips with different dimensions and with different number of oligos can be derived. For example, the depth of the cuts can range from about 0.3 mm to about 0.7 mm. In one preferred embodiment, the depth of the cuts is about 0.5 mm.

In one example of illustrating how the concept works, it is possible to study gene expression in an organism with a relatively small number of genes (e.g., yeast, approximately 5,000 genes) or one with a relatively larger number of genes (e.g., human or Arabidopsis, with approximately 30,000 genes). Each millichip can have not only one 70-mer per gene for that genome, but also a number of positive and negative control probes to check for quality control through the whole process, much like is done presently for other DNA chips. In terms of cost, since the cutting and sorting process is fairly easy, the cost per millichip at this point should be the cost associated with making the larger MAS derived microscope slide (approximately $100-200 in reagents per slide, ignoring cost of MAS instrument), divided by the number of resultant millichips, or approximately $0.50 to $4 per millichip depending on the genome.

The investigator can receive the millichip in the bottom of a 0.5 ml microfuge tube, to facilitate the remaining steps in this procedure. The next step is to apply labeled RNA to the millichip. Another improvement of the present invention is in decreasing the volume of hybridization solution. The reduction in hybridization solution volume improves the signal-to-noise ratio thereby decrease the background noise. This reduction in hybridization solution volume also results in significant cost reduction in the millichip process is that afforded by the fact that the millichip needs to be bathed in only approximately 2-10 microliters of solution, rather than the 300 microliters currently used to hybridize to uncut typical 2 cm² microscope slide surfaces. Thus, the $100-200 cost associated with labeling the RNA should be reducible by ratio of these volumes, or approximately about 30-fold to about 150-fold.

The volumes of prehybridization solution and hybridization solution can be adjusted accordingly for millichips of different sizes (dimensions). For example, if a square is 2× (twice) the 3 mm×3 mm size, then volumes noted of the prehybridization solution and hybridization solution are doubled.

After suitable washing to remove background hybridizing signal, also preferably performed by rinsing and centrifuging at low speed in a benchtop microfuge, the millichip is removed from the microfuge tube. This can be done in a variety of ways, e.g., with a forceps or by shaking. The millichip is then placed on a glass microscope slide, for viewing under a fluorescence microscope. In the examples described below, a normal visible light fluorescence microscope was used, but for additional resolution, a confocal microscope might be used. Both instruments are available in a modern laboratory.

Appropriate software can be used to allow one to convert the fluorescent images, which correspond to the amount of RNA hybridizing to each oligo used (e.g. each 70-mer), into a table of intensities. This software can be distributed to each investigator, together with the millichip, and would be usable with a variety of fluorescent images acquired, regardless of the microscope manufacturer.

It is contemplated that the methods of the present invention can find utility in the identification of genetic polymorphisms, SNP detection, and the like. For example, because of the inexpensive and ease of use of millichips, one skilled in the art could purchase a millichip for about $2-10 and perform the hybridizations at his/her benchtop that same morning, and see the results by the afternoon, without the need to work with a core facility or expensive instrumentation. By this means, any RNA or DNA sample that the investigator is interested in using, for any biological experiment, can readily be interrogated to find out if it is worth further exploration, by use of the millichip according to the present invention. For example, if the investigator is unsure of his/her quality of RNA isolation or storage, this millichip can be a fast and inexpensive means of performing a test. Even more importantly, the investigator can quickly narrow down the candidate genes involved in a biological pathway, from the, e.g., about 30,000 in the genome, to the handful that show the greatest change in a particular situation, very quickly. As just one example, if one skilled in the art is studying the onset of a new type of liver disease, a biopsied sample of liver can be used to isolate RNA and then one can determine which genes have greatly increased or decreased their expression under during the onset of the disease. Basically, any experiment that uses standard DNA chips can be done faster and cheaper, by an order of magnitude, with the millichip.

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

### EXAMPLES

### Example 1

In this example, the verification of hybridization of the complete chip was performed on an arrayWoRx scanner (Applied Precision, Issaquah, Wash.), and basically looked like a checkerboard. The chip was synthesized to contain a match and mismatch sequence and the Cy3probe that was hybridized to it was the reverse complement of the “match” sequence.

The oligonucleotide probes used were as follows. About 100 μM of each oligonucleotide were used. The oligonucleotides were: M-QC CY3, a Cy3 labeled 30-mer, which is 5′-GTCTGCCATGATGTATACATTGTGTGAGTT-3′ (SEQ ID NO:1); M-QC-RC, which is 5′-AACTCACACAATGTATACATCATGGCAGAC-3′ (SEQ ID NO:2); and MtM-QC-RC, which is 5′-AACTCACTCAATTTATAGATCACGGCAGAC-3′ (SEQ ID NO:3). These oligonucleotides were utilized to confirm the synthesis of DNA on the millichip surface through hybridization.

The oligonucleotides to be synthesized on chip were: M-QC-RC-2, which is 3′-CAGACGGTACTACATATGTAACACACTCAA-5′ (SEQ ID NO:4); and MtM-QC-RC-2, which is 3′-CAGACGGCACTAGATATTTAACTCACTCAA-5′ (SEQ ID NO:5). These oligonucleotides were utilized to allow for confirmation of sequence-specific hybridization.

The hybridization protocol was as follows. The prehybridization and hybridization solutions were prepared. The prehybridization solution consisted of: Herring Sperm DNA 10 mg/ml, 4 μl; Acetylated BSA 10 mg/ml, 20 μl; 2×MES Hybridization Buffer, 200 μl; and water, 176 μl, for a total of 400 μl. The hybridization solution consisted of: Cy3 labeled probe 100 nM, 30 μl; Herring Sperm DNA 10 mg/ml, 3 μl; Acetylated BSA 10 mg/ml, 15 μl; 2×MES Hybridization Buffer, 150 μl; water, 102 μl, for a total of 300 μl.

The prehybridization and hybridization solutions were transferred to a 95° C. heat block and heated for 5 minutes. Then the prehybridization and hybridization solutions were incubated at 45° C. for 5 minutes.

The prehybridization solution was spun in a microcentrifuge at >12,000×g for 10 minutes. The hybridization solution was kept incubating at 45° C. until prehybridization was occurring, for example by transferring to a 45° C. water bath until ready for use.

A scored microarray slide square (3 mm×3 mm×1 mm) was placed in a 0.6 ml eppendorf (i.e., microcentrifuge) tube. Forty μl of prehybridization solution was applied to the sample and incubated at 45° C. for 15 minutes.

The prehybridization solution was removed and 20 μl of prepared hybridization solution was applied per sample. The sample was incubated at 45° C. for 2 hours. The hybridization solution was then removed and immediately replace with 40 μl NS wash buffer. Pipetting up and down was performed to wash the sample. Fresh aliquot of NS wash buffer was added, and the rinses were repeated three times. The NS wash buffer was removed and 40 μl of 45° C. S wash buffer was added. Incubation at 45° C. for 30 minutes was performed, changing S wash buffer twice. The S wash buffer was pipetted off, and 40 μl NS wash buffer was added. The tubes were removed from the 45° C. incubator.

The NS wash buffer was then rinsed out, but small volume of NS wash buffer was left on the square until all samples have been processed. The purpose of this was to prevent artifacts noted with drying of the sample.

Ice-chilled FWB (Final Wash Buffer) was prepared, and the square was placed in FWB for 30 seconds. Then, the array was immediately dried for 2 minutes with Ar. FWB is a proprietary product of Roche-NimbleGen.

1×MES hybridization buffer consists of: 100 mM MES, 1 M Na⁺, 20 mM EDTA, 0.01% Tween-20. NSWB (or NS Wash Buffer) is Non-Stringent Wash Buffer (6×SSPE, 0.01% Tween-20). SWB (or S Wash Buffer) is Stringent Wash Buffer (100 mM MES salt and free acid solution, 0.1 M Na⁺, 0.01% Tween-20).

A Nikon fluorescence microscope was utilized for visualization of the Cy3-hybridized millichips and microarrays. Analysis of the hybridization results was performed through visual inspection and, when appropriate, quantitation of the intensity of the fluorescence signal.

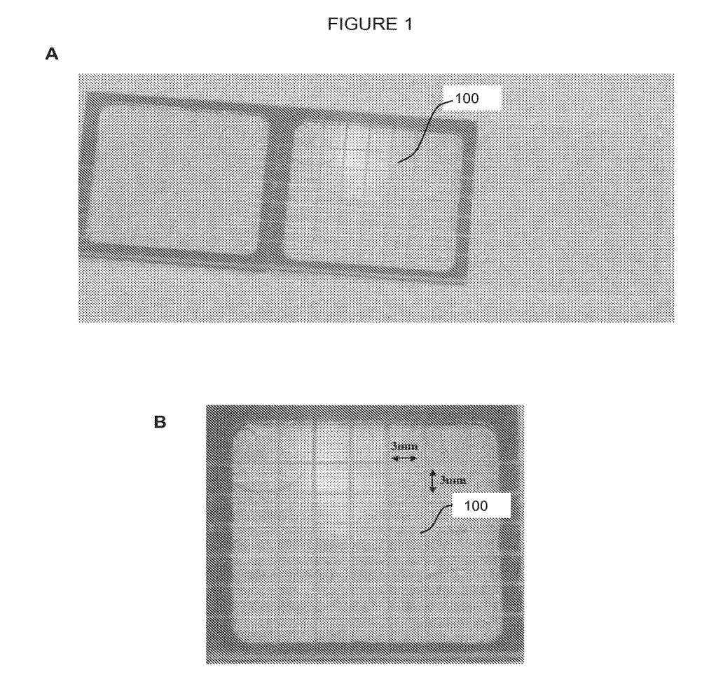

FIG. 1 is an image of 1 mm thick microscope slide used as solid substrate for array, and subdivided into 20 squares (i.e., 4×5). Affixed to the slide surface is a commercial hybridization chamber which encompasses the region upon which DNA has been synthesized. FIG. 1B is an enlarged portion 100 of the microscope slide in FIG. 1A, which shows the millichips (3 mm length×3 mm width×1 mm thickness) prior to disassembly.

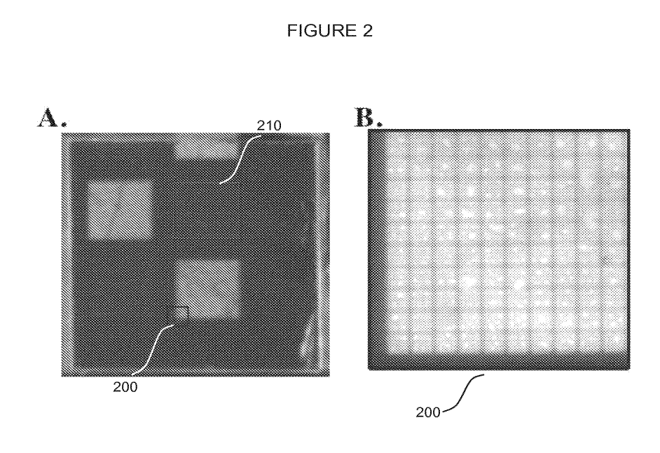

FIG. 2A is an image of a subdivided (1 mm×3 mm×3 mm) millichip and its hybridization. FIG. 2A is a fluorescent image of hybridized millichip (3 mm²). FIG. 2B is an enlarged portion 200 of the image in FIG. 2A. FIG. 2B is thus a 20× magnification image of one of the millichip arrays. In these hybridizations there was excellent resolution of individual features (see FIG. 2B) and there was high specificity (in FIG. 2A, the box 210 indicates the location of the negative hybridization control).

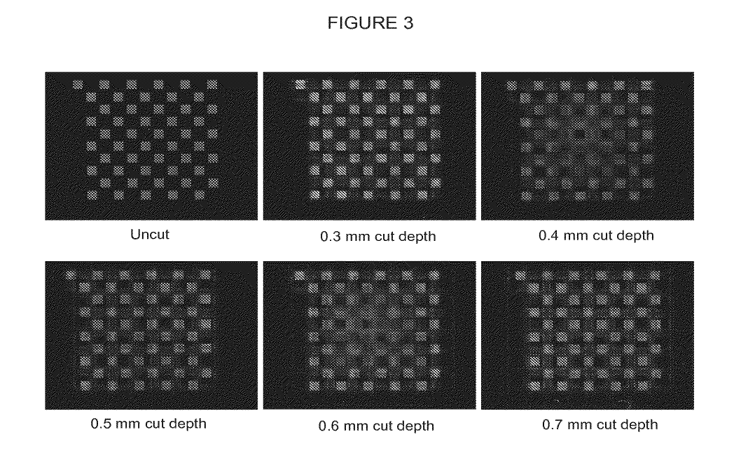

FIG. 3 is an image of hybridized arrays utilizing varying cut depths (0-0.7 mm cut depth) prior to subdivision. These arrays also have an increased number of subdivisions (grid of 9×12 squares) allowing for the production of 96 millichips.

### Example 2

A pre-scored slide was used in place of a regular microscope slide on the maskless array synthesizer (MAS). A simple procedure for aligning the slide was developed, and a reaction cell within the MAS was created such that the synthesis occurs within the pre-scored grid and away from the cuts. Briefly, this involves offsetting the slide within the reaction cell and then displaying a virtual mask containing a grid that is sized to fit within the score lines. Using a live image from the MAS camera the reaction cell is then moved until the displayed mask lines up with the scored grid. The slide is initially offset in the reaction cell to allow ‘image lock’ to be used. This system is in place to counteract any amount of movement of the reaction cell during the synthesis due to very minor disturbances and relies on a chrome mark on the reaction cell block. If the millichip slide is not offset this chrome mark falls on a scored section and renders the image lock system nonfunctional.

Once synthesis is complete the entire slide is deprotected. The synthesis surface of the slide (opposite of the pre-scored side) is then scored with a standard glass-scoring tool along the grid. This allows for clean breakage of the millichips along the pre-score lines and helps to prevent sheering or chipping of the synthesis surface.

The millichips are then hybridized in strips of PCR tubes. In this experiment 100 nM solutions of the fluorescent-labeled complements were used according to standard protocol, the same hybridization protocol used above. Either 5 microliters or 10 microliters of the hybridization solution was used and no noticeable difference was seen in the final images. Each millichip in this experiment contains a random and repeated set of sequences listed below as well as a unique marker sequence that is synthesized around the edge of each chip for alignment purposes.

TABLE 1
Probes used in hybridization experiments

| Probe sequence | Probe name | SEQ ID Numbers |
|---|---|---|
| -TGACAGCTCACCGAAGTCTC | MS1 | SEQ ID NO: 6 |
| TGACAGCTCCCCGAAGTCTC | MS1mm1 | SEQ ID NO: 7 |
| TGACAGCTCCACGAAGTCTC | MS1mm2 | SEQ ID NO: 8 |
| TGACAGCTCCAAGAAGTCTC | MS1mm3 | SEQ ID NO: 9 |
| -ATCGAAACCCACTGGCAGGG | MS2 | SEQ ID NO: 10 |
| ATCGAAACCAACTGGCAGGG | MS2mm1 | SEQ ID NO: 11 |
| ATCGAAACCATCTGGCAGGG | MS2mm2 | SEQ ID NO: 12 |
| ATCGAAACCATGTGGCAGGG | MS2mm3 | SEQ ID NO: 13 |
| -CCTGGAATACCGGAGCTACG | M53 | SEQ ID NO: 14 |
| CCTGGAATTCCGGAGCTACG | MS3mm1 | SEQ ID NO: 15 |
| CCTGGAATTCCTGAGCTACG | MS3mm2 | SEQ ID NO: 16 |
| CCTGGAATTCCTGATCTACG | MS3mm3 | SEQ ID NO: 17 |
| -AACAGTCGGAAACGTCGCCC | MS4 | SEQ ID NO: 18 |
| AACTGTCGGAAACGTCGCCC | MS4mm1 | SEQ ID NO: 19 |
| AACTGTCGGTAACGTCGCCC | MS4mm2 | SEQ ID NO: 20 |
| AACTGTCGGTAACGACGCCC | MS4mm3 | SEQ ID NO: 21 |
| -ACGTAGAGGCATCCCCTCTC | MS5 | SEQ ID NO: 22 |
| ACGTAGAGGTATCCCCTCTC | MS5mm1 | SEQ ID NO: 23 |
| ACGTAGAGGTATCCCATCTC | MS5mm2 | SEQ ID NO: 24 |
| ACGAAGAGGTATCCCATCTC | MS5mm3 | SEQ ID NO: 25 |

Table 1 lists the probe sequences that were used in the experiment. The probe sequences are indicated as MS1-MS5 and for each of the 5 probes there are 3 sequences with an increasing number of mismatched bases (1, 2, and 3 mismatches, which are shown as underlined nucleotides). After hybridization with fluorescent labeled complements for MS1-MS5 was performed, there was a strong signal for the exact match, decreasing signals for 1 mismatch and virtually no signal for sequences containing 3 mismatches. This shows that the millichip array hybridizations are selective and that there is a high level of resolution without spacer pixels within the array.

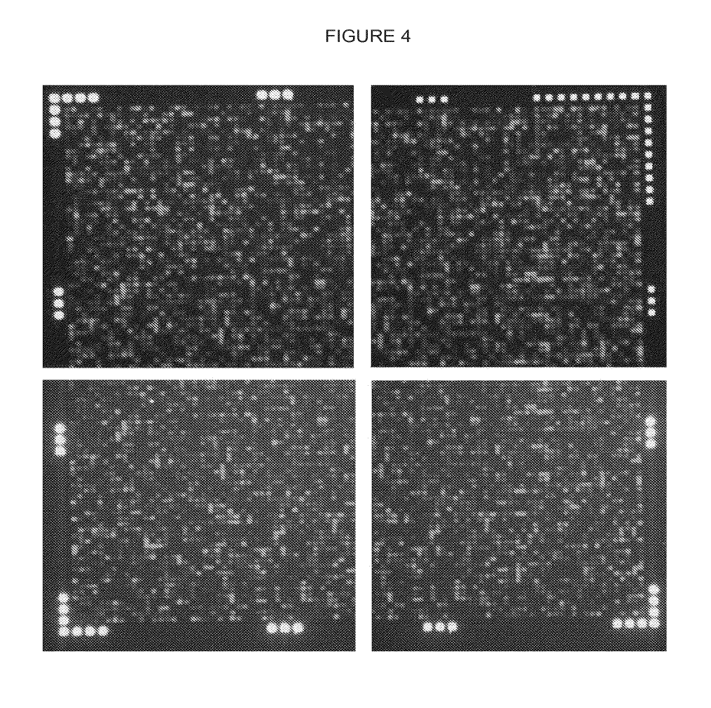

In this example, and as illustrated in FIG. 4, four fluorescent microscope images are required for each millichip. These images are put into the software which stitches them together, adjusts the alignment relative to the markers (bright dots in border areas) and records the normalized intensity of each pixel in the array.

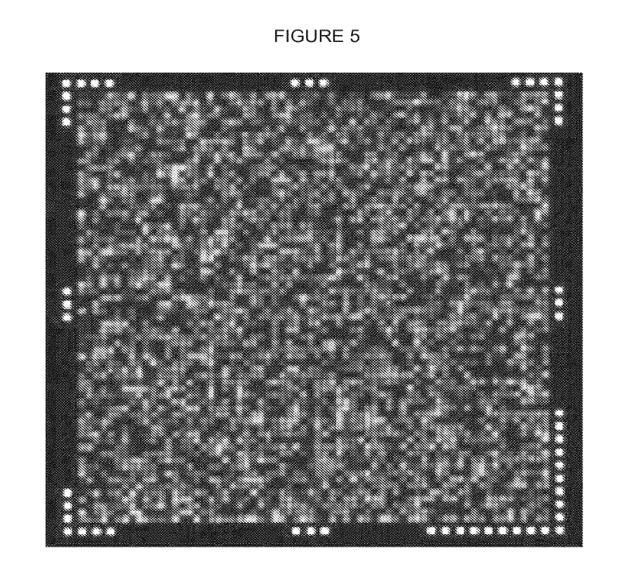

FIG. 5 shows a fluorescence scanner image of control slide millichip area. The control slide was not prescored (or broken) and was hybridized using standard hybridization procedures (hybridization chamber on the slide surface, 300 microliters solution, etc.).

Image analysis software can be used as follows. The software used can give intensities for each pixel, which can be correlated with the complementarity of the immobilized sequences with those of the assayed nucleotides. In some examples, due to the way in which the sequences are randomized in a given experiment, a script has to be constructed to attach the correct sequence to the intensity data. In other examples, this functionality can be a part of the finalized software. Once the intensity values are matched to the sequences, the control slide and millichip can be compared. In the example shown, a comparison of intensity values between the two images shows that the relative intensities do match up. Thus, it is possible to use appropriate software to get fluorescence intensity values, which correlate with hybridization intensity.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of bioengineering, molecular biology, molecular interactions, chemistry, and biology, are within the scope of this invention. All publications, patents, and patent applications cited herein are incorporated by reference in their entirety for all purposes.

What is claimed is:

1. An array of oligonucleotide probes immobilized on a solid support, the array comprising at least about 1,000 different oligonucleotide probes and no more than about 100,000 different oligonucleotide probes about 30 to about 100 nucleotides in length, wherein the oligonucleotide probes occupy separate known sites in the array, wherein the array has a density of at least about 500,000 oligonucleotide probes per 1 cm² solid support surface, wherein the volume of the solid support is between about 0.125 mm³ and about 30 mm³.

2. The array of claim 1, further comprising at least two sets of oligonucleotide probes: (1) a first set of oligonucleotide probes that is exactly complementary to a set of reference sequences; and (2) a second set of oligonucleotide probes that is identical to the first set of oligonucleotide probes but for at least one different nucleotide.

3. The array of claim 1, further comprising at least two sets of oligonucleotide probes: (1) a first set of oligonucleotide probes that is exactly complementary to a set of reference sequences; and (2) a second set of oligonucleotide probes that is a reverse-complement to the first set of oligonucleotide probes.

4. The array of claim 1 wherein the oligonucleotide probes are about 70 nucleotides in length.

5. The array of claim 1 wherein the density of the array is about 610,000 oligonucleotide probes per 1 cm² solid support surface.

6. The array of claim 1 wherein the volume of the solid support is about 8 mm³.

7. The array of claim 1 wherein the oligonucleotide probes are covalently attached to the solid support.

8. The array of claim 1 wherein the oligonucleotide probes are oligodeoxyribonucleotides.

9. The array of claim 1 wherein the solid support is an article that comprises at least one of a porous substrate, a non-porous substrate, a three-dimensional surface, a bead, and a planar surface.

10. A method for making an array of oligonucleotide probes, the method comprising immobilizing on a solid support an array that comprises at least about 1,000 different oligonucleotide probes and no more than about 100,000 different oligonucleotide probes about 30 to about 100 nucleotides in length, wherein the oligonucleotide probes occupy separate known sites in the array, wherein the array has a density of at least about 500,000 oligonucleotide probes per 1 cm² solid support surface, wherein the volume of the solid support is between about 0.125 mm³ and about 30 mm³; and cutting the solid support to obtain a DNA millichip.

11. The method of claim 10, further comprising immobilizing on the solid support an array that comprises at least two sets of oligonucleotide probes: (1) a first set of oligonucleotide probes that is exactly complementary to a set of reference sequences; and (2) a second set of oligonucleotide probes that is identical to the first set of oligonucleotide probes but for at least one different nucleotide.

12. The method of claim 10, further comprising immobilizing on the solid support an array that comprises at least two sets of oligonucleotide probes: (1) a first set of oligonucleotide probes that is exactly complementary to a set of reference sequences; and (2) a second set of oligonucleotide probes that is a reverse-complement to the first set of oligonucleotide probes.

13. The method of claim 10 wherein the oligonucleotide probes are about 70 nucleotides in length.

14. The method of claim 10 wherein the density of the array is about 610,000 oligonucleotide probes per 1 cm² solid support surface.

15. The method of claim 10 wherein the volume of the solid support is about 8 mm³.

16. The method of claim 10 wherein the oligonucleotide probes are covalently attached to the solid support.

17. The method of claim 10 wherein the oligonucleotide probes are oligodeoxyribonucleotides.

18. The method of claim 10 wherein the solid support is an article that comprises at least one of a porous substrate, a non-porous substrate, a three-dimensional surface, a bead, and a planar surface.

19. A method for detecting expression of a plurality of genes, the method comprising:
a) providing an array of oligonucleotide probes immobilized on a solid support, the array comprising at least about different 1,000 oligonucleotide probes and no more than about 100,000 different oligonucleotide probes about 30 to about 100 nucleotides in length, wherein the oligonucleotide probes occupy separate known sites in the array, wherein the array has a density of at least about 500,000 oligonucleotide probes per 1 cm² solid support surface, wherein the volume of the solid support is between about 0.125 mm³ and about 30 mm³;
b) hybridizing a labeled sample nucleic acid target to the array of oligonucleotide probes; and
c) measuring the label intensity to identify the level of gene expression for each of the labeled sample nucleic acid targets bound to complementary oligonucleotide probes.

20. The method of claim 19 wherein the plurality of genes comprises a genome.

21. The method of claim 19 wherein the hybridization is performed in about 5 μl to about 15 μl of hybridization solution.

22. The method of claim 19 wherein detecting the expression of a plurality of genes is used to identify at least one genetic polymorphism.


SEQUENCE LISTING (25 sequences)

SEQ ID NO: 1 - DNA, 30 bp, Artificial Sequence
  M-QC oligonucleotide
gtctgccatgatgtatacattgtgtgagtt

SEQ ID NO: 2 - DNA, 30 bp, Artificial Sequence
  M-QC-RC oligonucleotide
aactcacacaatgtatacatcatggcagac

SEQ ID NO: 3 - DNA, 30 bp, Artificial Sequence
  MtM-QC-RC oligonucleotide
aactcactcaatttatagatcacggcagac

SEQ ID NO: 4 - DNA, 30 bp, Artificial Sequence
  M-QC-RC-2 oligonucleotide
cagacggtactacatatgtaacacactcaa

SEQ ID NO: 5 - DNA, 30 bp, Artificial Sequence
  MtM-QC-RC-2 oligonucleotide
cagacggcactagatatttaactcactcaa

SEQ ID NO: 6 - DNA, 20 bp, Artificial Sequence
  Probe MS1
tgacagctcaccgaagtctc

SEQ ID NO: 7 - DNA, 20 bp, Artificial Sequence
  Probe MS1mm1
tgacagctccccgaagtctc

SEQ ID NO: 8 - DNA, 20 bp, Artificial Sequence
  Probe MS1mm2
tgacagctccacgaagtctc

SEQ ID NO: 9 - DNA, 20 bp, Artificial Sequence
  Probe MS1mm3
tgacagctccaagaagtctc

SEQ ID NO: 10 - DNA, 20 bp, Artificial Sequence
  Probe MS2
atcgaaacccactggcaggg

SEQ ID NO: 11 - DNA, 20 bp, Artificial Sequence
  Probe MS2mm1
atcgaaaccaactggcaggg

SEQ ID NO: 12 - DNA, 20 bp, Artificial Sequence
  Probe MS2mm2
atcgaaaccatctggcaggg

SEQ ID NO: 13 - DNA, 20 bp, Artificial Sequence
  Probe MS2mm3
atcgaaaccatgtggcaggg

SEQ ID NO: 14 - DNA, 20 bp, Artificial Sequence
  Probe MS3
cctggaataccggagctacg

SEQ ID NO: 15 - DNA, 20 bp, Artificial Sequence
  Probe MS3mm1
cctggaattccggagctacg

SEQ ID NO: 16 - DNA, 20 bp, Artificial Sequence
  Probe MS3mm2
cctggaattcctgagctacg

SEQ ID NO: 17 - DNA, 20 bp, Artificial Sequence
  Probe MS3mm3
cctggaattcctgatctacg

SEQ ID NO: 18 - DNA, 20 bp, Artificial Sequence
  Probe MS4
aacagtcggaaacgtcgccc

SEQ ID NO: 19 - DNA, 20 bp, Artificial Sequence
  Probe MS4mm1
aactgtcggaaacgtcgccc

SEQ ID NO: 20 - DNA, 20 bp, Artificial Sequence
  Probe MS4mm2
aactgtcggtaacgtcgccc

SEQ ID NO: 21 - DNA, 20 bp, Artificial Sequence
  Probe MS4mm3
aactgtcggtaacgacgccc

SEQ ID NO: 22 - DNA, 20 bp, Artificial Sequence
  Probe MS5
acgtagaggcatcccctctc

SEQ ID NO: 23 - DNA, 20 bp, Artificial Sequence
  Probe MS5mm1
acgtagaggtatcccctctc

SEQ ID NO: 24 - DNA, 20 bp, Artificial Sequence
  Probe MS5mm2
acgtagaggtatcccatctc

SEQ ID NO: 25 - DNA, 20 bp, Artificial Sequence
  Probe MS5mm3
acgaagaggtatcccatctc